(19) United States
(12) Patent Application Publication
(10) Pub. No.: US 2008/0281195 A1
(43) Pub. Date: Nov. 13, 2008

(54) System and method for planning LV lead placement for cardiac resynchronization therapy

(75) Inventor: Andreas Heimdal, Oslo (NO)

Correspondence Address: DEAN D. SMALL;THE SMALL PATENT LAW GROUP LLP, 225 S. MERAMEC, STE. 725T, ST. LOUIS, MO, 63105, US

(73) Assignee: GENERAL ELECTRIC COMPANY

(21) Appl. No.: 11/801,271

(22) Filed: May 9, 2007

(51) Int. Cl. A61B 8/00

(52) U.S. Cl. 600/437

(57) ABSTRACT

An ultrasound system comprises a memory for storing patient study data associated with segments of a patient's left ventricle (LV). The patient study data comprises at least a first asynchrony study and at least one of a first viability study and a first regional function study. A cardiac resynchronization therapy (CRT) lead placement planning module compares at least a portion of the patient study data for each of the segments. An output device indicates at least one location for placement of an LV lead within one of the segments of the patient's LV during a CRT procedure based on the comparison of the at least a portion of the patient study data for each of the segments.

### BACKGROUND OF THE INVENTION

This invention relates generally to cardiac resynchronization therapy (CRT), and more specifically, to determining the placement positions of the lead within the left ventricle (LV) prior to the CRT implantation procedure.

CRT is a device therapy that is used to treat congestive heart failure. A small device is placed under a patient's skin to deliver electrical signals to the patient's heart. CRT typically involves three pacing leads, one of which is placed on the left ventricle through the coronary veins, or alternatively by surgical epicardial placement. The lead is typically placed in a standard location. In some cases, an alternative location is selected if it is not possible to place the lead in the standard location due to the anatomy of the coronary veins. Each patient has different pathology such that a patient's heart tissue has unique characteristics directed to viability and delay in motion. Therefore, positioning the lead in the left ventricle based on a standard or convenient location may not achieve the best result and a subsequent procedure may be needed to reposition the lead. Also, if the tissue at the placement site is non-functional, the lead may have no effect.

In other cases, the lead may be placed in more than one location within the left ventricle during the implantation procedure. Measurements of the heart function are compared during the procedure while the lead is in the different locations. This increases the length of time required for the surgery as well as the complexity.

Therefore, a need exists for determining one or more implantation positions for the lead within the left ventricle that decreases the complexity of the procedure as well as provides an optimal resynchronization result.

### BRIEF DESCRIPTION OF THE INVENTION

In one embodiment, an ultrasound system comprises a memory for storing patient study data associated with segments of a patient's left ventricle (LV). The patient study data comprises at least a first asynchrony study and at least one of a first viability study and a first regional function study. A cardiac resynchronization therapy (CRT) lead placement planning module compares at least a portion of the patient study data for each of the segments. An output device indicates at least one location for placement of an LV lead within one of the segments of the patient's LV during a CRT procedure based on the comparison of the at least a portion of the patient study data for each of the segments.

In another embodiment, a method for recommending placement for a left ventricle (LV) lead prior to a CRT procedure comprises identifying a first asynchrony study associated with a patient. The first asynchrony study comprises at least delay data with respect to each segment within an LV of the patient. One of a first viability study and a first regional function study is identified wherein the first viability study comprises viability data with respect to each of the segments and the first regional function study comprises regional function data with respect to each of the segments. The segments are compared based on the delay data and at least one of the viability data and the regional function data. At least one of the segments and an associated recommendation with respect to placement of an LV lead within the patient's LV during a CRT procedure is output.

In yet another embodiment, a machine readable medium or media having instructions recorded thereon that are configured to instruct a computer having a processor, a display and a memory to identify LV segments for placement of an LV lead during a CRT procedure. The LV segments are identified based on a degree of delay and a degree of viability. The computer readable medium or media further comprises instructions to form an output based on the degree of delay and the degree of viability of the LV segments.

### BRIEF DESCRIPTION OF THE DRAWINGS

FIG. 1 illustrates a block diagram of an ultrasound system formed in accordance with an embodiment of the present invention.

FIG. 2 illustrates a miniaturized ultrasound system formed in accordance with an embodiment of the present invention.

FIG. 3 illustrates a method for determining and providing left ventricle (LV) lead placement recommendations prior to a cardiac resynchronization therapy (CRT) implantation procedure in accordance with an embodiment of the present invention.

FIG. 4 illustrates a general bull's eye plot divided into 16 segments in accordance with an embodiment of the present invention.

FIG. 5 illustrates an example of using a bull's eye plot of a 16 segment model of the LV to display LV lead placement recommendations for CRT in accordance with an embodiment of the present invention.

FIG. 6 illustrates a 3D surface model that may be used to display LV lead placement recommendations for CRT in accordance with an embodiment of the present invention.

### DETAILED DESCRIPTION OF THE INVENTION

The foregoing summary, as well as the following detailed description of certain embodiments of the present invention, will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or random access memory, hard disk, or the like). Similarly, the programs may be stand alone programs, may be incorporated as subroutines in an operating system, may be functions in an installed software package, and the like. It should be understood that the various embodiments are not limited to the arrangements and instrumentality shown in the drawings.

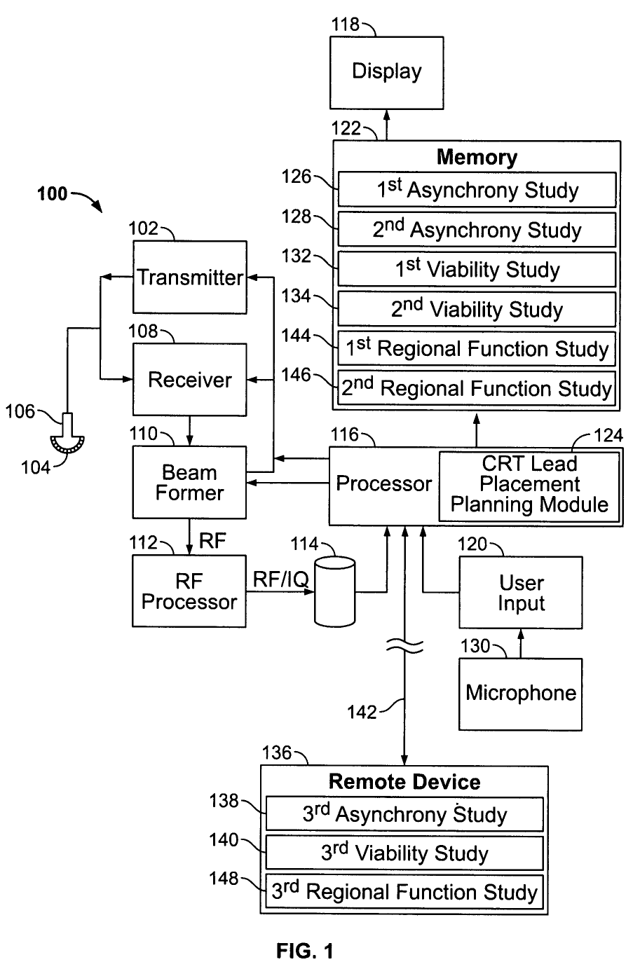

FIG. 1 illustrates a block diagram of an ultrasound system 100. The ultrasound system 100 includes a transmitter 102 that drives transducer elements 104 within a probe 106 to emit pulsed ultrasonic signals into a body. The ultrasonic signals or transmit beams are back-scattered from structures in the body, like blood cells or muscular tissue, to produce echoes or return beams that return to the transducer elements 104. The returning echoes are converted by the transducer elements 104 back to electrical energy that is received by a receiver 108. The received signals are passed through a beamformer 110 that performs beamforming (combining the transducer element signals to perform steering and focusing of the beam) and outputs an RF signal. The RF signal then passes through an RF processor 112. Alternatively, the RF processor 112 may include a complex demodulator (not shown) that demodulates the RF signal to form IQ data pairs representative of the echo signals. The RF or IQ signal data may then be routed directly to an RF/IQ buffer 114 for temporary storage.

A user input 120 may be used to control operation of the ultrasound system 100, including, to control the input of patient data and scan parameters, to select a cardiac resynchronization therapy (CRT) lead placement tool, select and/or change how the preferred and/or optimal left ventricle (LV) lead placements are displayed, and may also include using voice commands provided via a microphone 130. Other various embodiments such as a set of user controls may be configured for controlling the ultrasound system 100 and may be provided, for example, as part of a touch screen or panel, and as manual inputs, such as user operable switches, buttons, and the like. The set of user controls may be manually operable or voice operated.

The ultrasound system 100 also includes a processor 116 to process the acquired ultrasound information (i.e., RF signal data or IQ data pairs) and prepare frames of ultrasound information for display on display 118. The processor 116 is adapted to perform one or more processing operations according to a plurality of selectable ultrasound modalities on the acquired ultrasound information. Acquired ultrasound information may be processed in real-time during a scanning session as the echo signals are received.

Prior to a CRT procedure, a patient typically undergoes a number of different procedures that are performed to determine heart function. The procedures may be previously acquired, such as during other diagnostic or routine examinations, or may be specifically performed for use with the CRT procedure. For example, tools exist for determining degrees of asynchrony or timing, viability, and function of the heart tissue. Asynchrony or asynchronous timing refers to different segments of the LV that do not contract at the same time. An asynchrony parameter that is typically measured is delay in time to peak velocity. Another asynchrony parameter is delay in time to minimum regional volume. Viability refers to whether the portion (or segment) of the LV is alive, and how much is alive, as muscle may be damaged or dead due to previous infarct. Regional function refers to the degree of functionality of the segment, typically the segment's ability to contract and the degree of contraction, motion or wall thickening.

The procedures or studies typically divide the LV into a number of segments, such as 16, 17 or 18 segments that may be named based on a location along the long axis of the left ventricle, such as basal, mid-level or mid-cavity, and apical, and on a circumferential position typically given by the cardiac wall name (septal, anterior septal, anterior, lateral, posterior, and inferior wall). The data may be 2D, 3D, 4D and/or multi-plane datasets and thus is not limited to any particular dataset or acquisition type. The data may be acquired using various techniques such as, for example, echocardiography, cardiac MRI, SPECT (as acquired using Nuclear Medicine) or PET scans.

One example of an ultrasonic asynchrony study is Tissue Synchronization Imaging (TSI), which is a tool that automatically measures time to peak velocity using tissue Doppler imaging. For example, TSI is based on 2D/multi-plane tissue Doppler. Each segment is assigned a number in milliseconds wherein the highest number is associated with the segment having the greatest delay. Another example of asynchrony studies are Regional Volumes that automatically measure time to minimum regional volume based on endocardial border detection in four dimensional (4D) ultrasound imaging. Each asynchrony study may assign a number or rating, for example, to each of the LV segments, or may rate the LV segments based on the highest to lowest delay in time to peak velocity.

An example of an ultrasonic viability study is stress echocardiography (e.g. low-dose Dobutamine stress echocardiography). Examples from other modalities include, but are not limited to, Thallium-SPECT (acquired using Nuclear Medicine), FDG-PET and cardiac MRI (CMR). The definition of a viable segment depends, at least in part, on the method used. Turning to stress echocardiography, viability is typically defined as improved wall motion during stress or a bi-phasic response, such as an improvement at low dose, but worsening at high dose. The operator may visually estimate or score wall motion to associate a level of regional function with a segment. The operator typically selects segments and/or inputs data associated with each of the segments. In PET, a discrepancy between flow and metabolism may be used to evaluate viability. For example, FDG-PET may be used to quantify metabolism, while one or more other tracers may be used to quantify flow (perfusion).

In some cases, studies may estimate regional function. Quantitative Strain imaging, such as Automated Function Imaging (AFI), may automatically estimate regional strain based on speckle tracking in ultrasound images. For example, AFI is based on 2D image analysis that may be detected in apical slices. In one embodiment, a regional function study may be performed with AFI at resting conditions. Segments with low or opposite sign strain at rest may be non-viable, and may therefore not be the first choice for LV lead placement. Other strain imaging to measure deformation of tissue may also be used, such as MRI tagging techniques. If the peak systolic strain is close to zero or even in opposite direction than normal, and does not respond to stress induced by, for example Dobutamine infusion or physical exercise, the segment is probably not viable. Other automated and/or operator defined wall motion analysis may be used.

It should be understood that other asynchrony, viability and regional function studies may be used and are not limited to the modalities discussed previously. The LV segment data may also in some cases be entered manually based on one or more procedures that do not automatically generate the LV segment data.

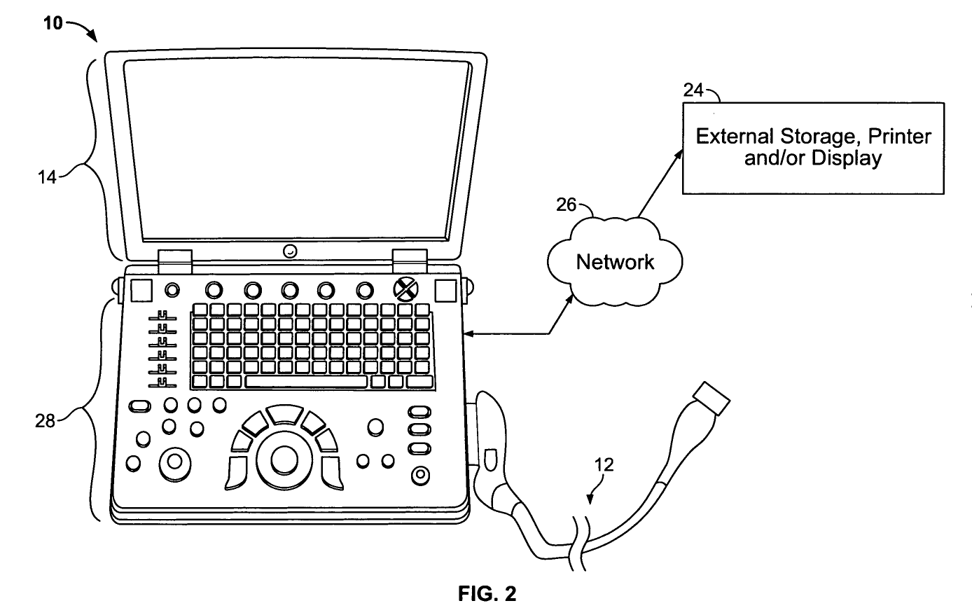

Referring again to FIG. 1, a CRT lead placement planning module 124 may determine one or more optimal or recommended placements or locations of the LV lead based on the previously acquired and processed asynchrony and viability or functional data specific to the patient. The CRT lead placement planning module 124 may be implemented in hardware or software, or a combination thereof. The CRT lead placement planning module 124 accesses data specific to the patient that may be stored in memory 122 on the ultrasound system 100, such as one or more of first and second asynchrony studies 126 and 128, first and second viability studies 132 and 134, and first and second regional function studies 144 and 146. The CRT lead placement planning module 124 may also access patient data that is stored remote from the ultrasound system 100. For example, the system 100 may be interconnected with a network 142 that may be hardwired or wireless and the patient diagnostic data may be located on a remote device 136. The remote device 136 may be a server, workstation, or another diagnostic imaging system such as another ultrasound system (e.g. system 10 of FIG. 2), Nuclear Medicine, PET, CT, MRI, and the like. It should be understood that although a single remote device 136 is illustrated, the system 100 may access asynchrony, viability and/or regional function study data from more than one remote device. One or more of a third asynchrony study 138, a third viability study 140, and a third function study 148 may be stored on the remote device 136.

It should be understood that the functionality discussed with respect to the system 100 is not limited to any ultrasound system type. For example, the system 100 may be housed within a cart-based system or may be implemented in a smaller, portable system as discussed in FIG. 2.

FIG. 2 illustrates a miniaturized ultrasound system 10 having a probe 12 configured to acquire ultrasonic data. As used herein, “miniaturized” means that the ultrasound system is a handheld or hand-carried device or is configured to be carried in a person's hand, pocket, briefcase-sized case, or backpack. For example, the ultrasound system 100 may be a hand-carried device having a size of a typical laptop computer, for instance, having dimensions of approximately 2.5 inches in depth, approximately 14 inches in width, and approximately 12 inches in height. The ultrasound system 10 may weigh about ten pounds, and thus is easily portable by the operator. An integrated display 14 (e.g., an internal display) is also provided and is configured to display a medical image.

The ultrasonic data may be sent to external device 24 via a wired or wireless network (or direct connection, for example, via a serial or parallel cable or USB port) 26. In some embodiments, external device 24 may be a computer or a workstation having a display. Alternatively, external device 24 may be a separate external display or a printer capable of receiving image data from the hand carried ultrasound imaging system 10 and of displaying or printing images that may have greater resolution than the integrated display 14.

A user interface 28 (that may also include integrated display 14) is provided to receive commands from an operator. The acquired image data may be acquired in a higher resolution than that displayable on the integrated display 14.

As another example, the ultrasound system 10 may be a pocket-sized ultrasound system. By way of example, the pocket-sized ultrasound system may be approximately 2 inches wide, approximately 4 inches in length, and approximately 0.5 inches in depth and weigh less than 3 ounces. The pocket-sized ultrasound system may include a display, a user interface (i.e., keyboard) and an input/output (I/O) port for connection to the probe (all not shown). It should be noted that the various embodiments may be implemented in connection with a miniaturized ultrasound system having different dimensions, weights, and power consumption.

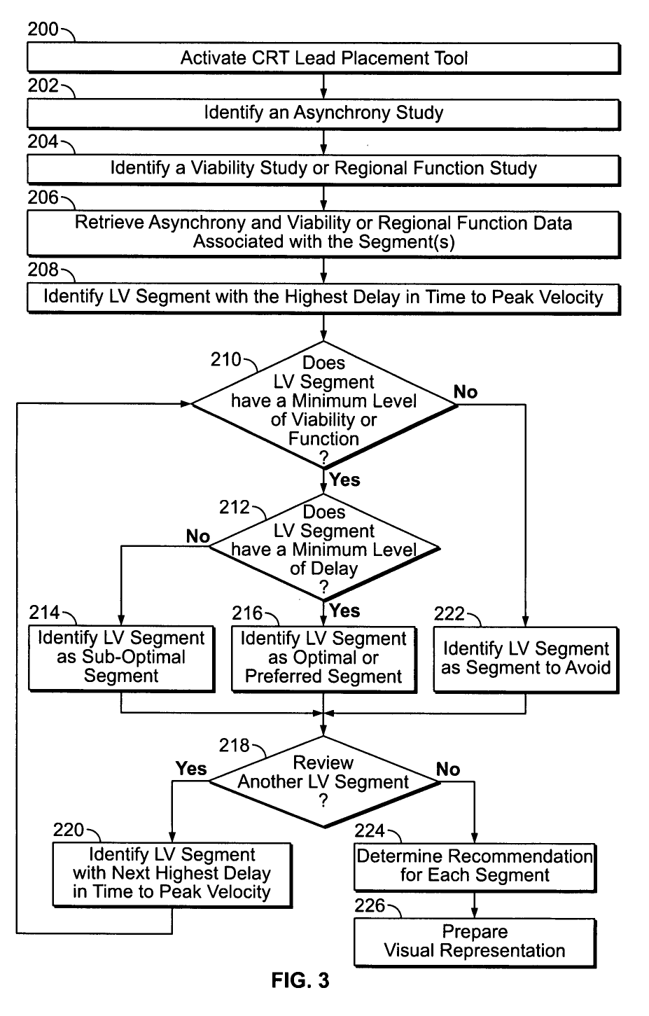

FIG. 3 illustrates a method for determining and providing LV lead placement recommendations prior to the CRT implantation procedure. Knowing the LV lead placement recommendations prior to the CRT implantation procedure may facilitate easy planning and preparation for the procedure. Also, by predicting one or more optimal or recommended placement positions for the LV lead, the success rate of CRT may be increased.

In general, previous results have indicated that CRT may be most beneficial when the lead is placed in the LV segment that has the highest delay in time to peak velocity. However, if the LV segment is non-viable, the pacing will have little or no effect on the segment and thus will not have the desired effect on the heart. Therefore it is desirable to identify candidate segments that have high delay, or some measurable level of delay, as well as a high likelihood of being viable. The information is then combined into one display for easy review and analysis by the operator prior to the CRT procedure. Although the method is described primarily in terms of the system 100 of FIG. 1, it should be understood that the functionality of the CRT lead placement planning module 124 may also be provided on the system 10 of FIG. 2, as well as in a remote system such as a workstation or processing station or the remote device 136 of FIG. 1. The method also may be used with other types of diagnostic imaging systems.

At 200, the operator may activate the CRT lead placement planning module 124 by making a selection on a protocol menu, such as accessed with a button or soft key on the user input 120 or displayed on a menu on the display 118. The operator also identifies the patient, such as with a unique patient code and/or patient name.

At 202, the CRT lead placement planning module 124 may prompt the operator to identify an asynchrony study associated with the patient. For example, the asynchrony study may be one of the first and second asynchrony studies 126 and 128 in the memory 122 of the system 100 (as shown in FIG. 1). In this example, the first and second asynchrony studies 126 and 128 may be displayed on the display 118 such that the operator may select the desired study. The asynchrony study may also be stored remotely from the system 100, such as the third asynchrony study 138 stored at the remote device 136. In this case, the operator may browse to the remote device 136, or optionally, the CRT lead placement planning module 124 may search identified and/or available network locations to identify studies associated with the patient.

At 204, the CRT lead placement planning module 124 may prompt the operator to identify and/or select a viability study, such as one of the first, second and third viability studies 132, 134 and 140 or a regional function study, such as one of the first, second and third regional function studies 144, 146 and 148. In the following example, the user has identified and/or selected the first asynchrony study 126 and the first viability study 132.

At 206, the CRT lead placement planning module 124 accesses the first asynchrony study 126 and the first viability study 132 to retrieve the data associated with each of the segments within the LV. As discussed previously, each of the asynchrony, viability and regional function studies assigns an indication, such as a number within a range of numbers, to each of the segments. The number indicates a measurement or degree associated with the parameter that the particular study is measuring. It should be understood that the CRT lead placement planning module 124 may access all of the segment data from each study at one time, or may access the data for each segment individually during the method. By way of example, a degree of viability may be related to a level of scored wall motion (such as comparison of normal segments to hypokinetic segments, etc.), and a degree of function may be related to an amount of peak negative systolic strain. In FDG-PET, the degree of viability may be related to the degree of mismatch between flow and metabolism. It should be understood that different parameters may be used to provide a degree of viability and/or function.

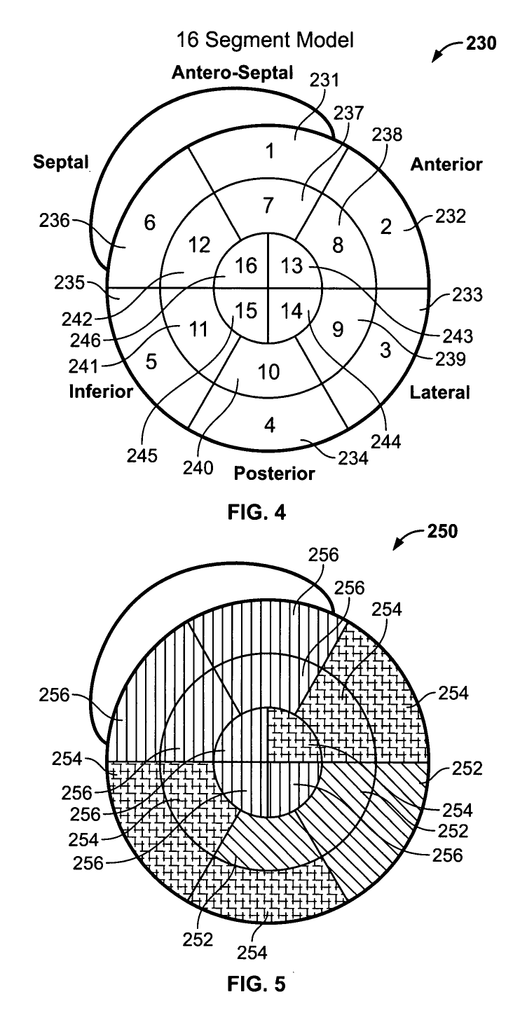

FIG. 4 illustrates a general bull's eye plot 230 divided into 16 segments. The bull's eye plot is a schematic overview of the LV. As discussed previously, the LV may be divided into other numbers of segments, such as 17 or 18 segments (not shown). Each of the segments is numbered, namely first segment 231, second segment 232, third segment 233, fourth segment 234, fifth segment 235, sixth segment 236, seventh segment 237, eighth segment 238, ninth segment 239, tenth segment 240, eleventh segment 241, twelfth segment 242, thirteenth segment 243, fourteenth segment 244, fifteenth segment 245 and sixteenth segment 246.

Returning to FIG. 3, at 208 the CRT lead placement planning module 124 may compare the delay values for each of the first through sixteenth segments 231-246 to identify the segment within the first asynchrony study 126 that has the highest delay in time to peak velocity. The CRT lead placement planning module 124 may optionally identify an order of the first through sixteenth segments 231-246 from highest delay to least delay, and may also identify segments that do not experience any delay.

At 210, the CRT lead placement planning module 124 accesses the first viability study 132 to determine whether the segment identified at 208 has a minimum level of viability or function. The minimum level of viability may be based on, for example, a predetermined degree of viability or on an operator preference. If there is a minimum level of viability or function, the method passes to 212 where the CRT lead placement planning module 124 compares the delay in time to peak velocity to a minimum level of delay. The minimum level of delay may be predetermined so that segments that have little or no delay, but also have viability or function greater than the minimum levels at 210, are not identified as preferred over segments that have a higher delay. If the segment has little or no delay, the method passes to 214 and the CRT lead placement module identifies the particular segment as a sub-optimal segment for LV lead positioning. If the segment has at least the minimum level of delay, the method passes to 216 where the CRT lead placement planning module 124 identifies the particular segment as a preferred or optimal segment for LV lead positioning. In many cases, it may be desirable to review all of the segments, and the method passes to 218 from both 214 and 216. At 218, if more segments are to be reviewed, the method passes to 220 to identify the segment that has the next highest delay in time to peak velocity. After identifying the segment, the method returns to 210.

If at 210 the CRT lead placement planning module 124 determines that the segment identified at 208 does not have a minimum level of viability or function, the method passes to 222 and the CRT lead placement planning module 124 identifies the segment as a segment to avoid. As discussed previously, placing the LV lead on a segment that is not viable may have little or no impact. The method then passes from 222 to 218 to determine if any of the first through sixteenth segments 231-246 remains to be reviewed and/or categorized.

When all of the first through sixteenth segments 231-246 have been reviewed for placement of the LV lead during CRT, the method passes to 224. At 224 the CRT lead placement planning module 124 may determine a recommendation for each of the first through sixteenth segments 231-246. In one embodiment, the CRT lead placement planning module 124 may prioritize the first through sixteenth segments 231-246 individually, indicating the segments from most preferred position to least preferred position. Any segments that do not have the minimum level of viability may be indicated such that the physician knows that the LV lead should not be positioned within the segment.

In another embodiment, the CRT lead placement planning module 124 may divide, categorize, sort or otherwise prioritize the first through sixteenth segments 231-246 into one or more groups, such as a Preferred Position Group, a Sub-Optimal Position Group, and a Position to Avoid Group. The Position to Avoid Group may be used to indicate segments that have less than the minimum level of viability. If no segments have viable tissue, then all of the first through sixteenth segments 231-246 may be indicated within the Position to Avoid group. In some embodiments, such as when a regional function study was used, one or more of the segments with no regional function may have viable tissue, and thus CRT may still be a useful therapy.

If none of the first through sixteenth segments 231-246 are identified for the Position to Avoid Group, then all of the first through sixteenth segments 231-246 may be indicated as having viable tissue and may be in the Preferred Position Group or Sub-Optimal Position Group. Optionally, segments having no delay and having viable tissue may be indicated in a different group. The segments may be divided into the Preferred Position Group and the Sub-Optimal Position Group based on degree of delay, degree of viability and/or function, or may be divided based on displaying an approximately equal number of segments within each group. Optionally, if the optimum placement is in a segment of the interventricular septum, which is the wall that separates the right and left ventricles, the output may indicate that it may be advantageous to perform right ventricle pacing only as a cost savings compared to CRT.

When a recommendation has been determined for each of the reviewed segments, at 226 the CRT lead placement planning module 124 prepares an output or visual representation for the operator. The output may also be stored in the memory 122 and may be associated with the patient's file. The visual representation may be a bull's eye plot, a surface model, list of the first through sixteenth segments 231-246 with corresponding recommendations, combination of the output with venogram of coronary veins, a chart, spreadsheet and the like.

The discussion above with respect to FIG. 3 assumes that the first asynchrony study 126 and the first viability study 132 have segmented the LV into the same number of segments. In one embodiment, the CRT lead placement planning module 124 may prompt the operator to select asynchrony and viability studies that have segmented the LV into the same number of segments. In another embodiment, the method of FIG. 3 may determine the recommendations for each of the segments when the LV is divided into different numbers of segments. In this case, the segments in the asynchrony and viability studies may be initially correlated to one another and/or the results may be displayed superposed on one another.

In another embodiment, the CRT lead placement planning module 124 may determine recommendations for each of the first through sixteenth segments 231-246 based on multiple studies, such as the first and second asynchrony studies 126 and 128 and one or both of the first and second viability studies 132 and 134. If the results are the same from all of the studies used, the CRT lead placement planning module 124 may form the output as previously discussed. However, if the results vary from one study to another, the CRT lead placement planning module 124 may form an additional output to draw the operator's attention to the differences.

FIG. 5 illustrates an example of using a bull's eye plot 250 of a 16 segment model of the LV to display LV lead placement recommendations for CRT. Referring also to the bull's eye plot 230 of FIG. 4, the CRT lead placement planning module 124 has grouped the first through sixteenth segments 231-246 into Preferred Positions, Sub-Optimal Positions and Positions to Avoid by indicating groups of segments in different colors, shades of gray, patterns or textures, and the like. The third, ninth, and tenth segments 233, 239 and 240 are displayed in a first color 252 that indicates segments that are in the optimal or Preferred Position for LV lead placement. The second, fourth, fifth, eighth, eleventh and thirteenth segments 232, 234, 235, 238, 241, and 243 are displayed in a second color 254 that indicates segments that are in the Sub-Optimal Position for LV lead placement. The first, sixth, seventh, twelfth, fourteenth, fifteenth, and sixteenth segments 231, 236, 237, 242, 244, 245 and 246 are displayed in a third color 256 that indicates segments that are in Positions to Avoid, and thus the LV lead should, if possible, not be placed in these segments.

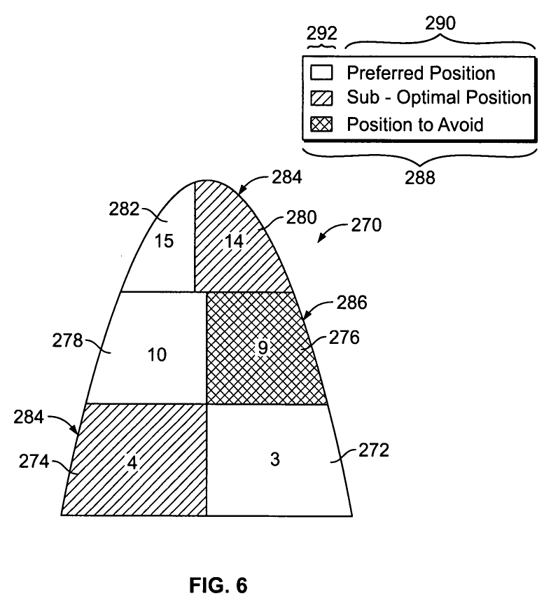

FIG. 6 illustrates a 3D surface model 270 that may be used to display LV lead placement recommendations for CRT. In this example, the third, fourth, ninth, tenth, fourteenth and fifteenth segments 272, 274, 276, 278, 280 and 282 are illustrated. The groups of segments may each be indicated differently, such as with a different color as discussed in FIG. 5, a different grayscale level or pattern, or a different pattern as displayed in FIG. 6. A legend 288 associating recommendations 290 with display indicators 292 may be provided. The third, tenth and fifteenth 272, 278 and 282 may be Preferred Positions, and may be indicated without a pattern along the surface of the model 270. The fourth and fourteenth segments 274 and 280 may be indicated with a first pattern 284 to indicate the segments that are Sub-Optimal Positions. The ninth segment 276 may be indicated with a second pattern 286 to indicate the segments that are Positions to Avoid.

A chart (not shown) may also be provided alone or with one of the visual indications of FIGS. 5 and 6 to display the CRT LV lead placement recommendations. The chart may list each of the first through sixteenth segments 231-246 (and seventeenth or eighteenth segments as appropriate) followed by the LV lead placement recommendation. Alternatively, the CRT lead placement planning module 124 may group the segments together as discussed previously, or may list the first through sixteenth segments 231-246 from most optimal to least optimal. The segments that are not viable may be highlighted such that the operator is aware that the LV pacing lead should not be placed in the segment. Optionally, the CRT lead placement planning module 124 may also list or display the specific data for each of the first through sixteenth segments 231-246 from the applicable asynchrony study and viability or function study.

A technical effect of at least one embodiment is to identify optimal or preferred candidate segments for LV lead placement prior to a CRT implantation procedure. The segments within the LV are automatically evaluated based on diagnostic studies of the patient, such as an asynchrony study and one of a viability and regional function study. The segments are evaluated to identify which, if any, will likely provide an optimal or preferred result (e.g. the segment is viable and has a greatest and/or measurable delay in time to peak velocity). The segments may be ordered or grouped, such as to identify a preferred or sub-optimal position, or a position to avoid. The result for each segment is then output, such as in a list, a bull's eye plot, 3D model or with other visual indication.

It is to be understood that the above description is intended to be illustrative and not restrictive. For example, the above-described embodiments (and/or aspects thereof) may be used in combination with each other. In addition, many modifications may be made to adapt a particular situation or material to the teachings of the invention without departing from its scope. While the dimensions and types of materials described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. § 112, sixth paragraph, unless and until such claim limitations expressly use the phrase “means for” followed by a statement of function void of further structure.

What is claimed is:

1. An ultrasound system, comprising:
a memory for storing patient study data associated with segments of a patient's left ventricle (LV), the patient study data comprising at least a first asynchrony study and at least one of a first viability study and a first regional function study;
a cardiac resynchronization therapy (CRT) lead placement planning module for comparing at least a portion of the patient study data for each of the segments; and
an output device for indicating at least one location for placement of an LV lead within one of the segments of the patient's LV during a CRT procedure based on the comparison of the at least a portion of the patient study data for each of the segments.

2. The system of claim 1, wherein the output device comprises a display for displaying at least one of a list, a chart, a bull's eye plot, and a 3D model for displaying an association between at least one of the segments and the at least one location.

3. The system of claim 1, wherein the first asynchrony study comprises timing data that associates each of the segments with a delay in time to peak velocity, the CRT lead placement planning module identifying a first segment from within the segments that has a highest delay in time to peak velocity, the CRT lead placement planning module determining if the first segment exceeds at least one of a minimum level of viability and a minimum level of function based on the first viability study and the first regional function study, respectively.

4. The system of claim 1, wherein the CRT lead placement planning module associates each of the segments with a group, wherein the group comprises one of a preferred position, a sub-optimal position and a position to avoid, the output device identifying the segments in the preferred position with a first indication, the segments in the sub-optimal position with a second indication, and the segments in the position to avoid with a third indication, the first, second and third indications being different with respect to each other.

5. The system of claim 1, wherein the CRT lead placement planning module further comprises identifying a first segment having a highest delay in time to peak velocity, the CRT lead placement planning module further comparing a level of viability of the first segment to a minimum level of viability, and the output device further comprising outputting a recommendation to place the LV lead within the first segment when the first segment exceeds the minimum level of viability and a recommendation to avoid the first segment when the first segment has less than the minimum level of viability.

6. The system of claim 1, wherein the output device comprises a display for displaying a bull's eye plot based on the LV, the at least one location for placement of an LV lead comprising indicating the segments on the bull's eye plot in one of different colors, different shades of gray, and different patterns.

7. A method for recommending placement for a left ventricle (LV) lead prior to a cardiac resynchronization therapy (CRT) procedure, the method comprising:
identifying a first asynchrony study associated with a patient, the first asynchrony study comprising at least delay data with respect to each segment within an LV of the patient;
identifying one of a first viability study and a first regional function study, the first viability study comprising viability data with respect to each of the segments and the first regional function study comprising regional function data with respect to each of the segments;
comparing the segments based on the delay data and at least one of the viability data and the regional function data; and
outputting at least one of the segments and an associated recommendation with respect to placement of an LV lead within the patient's LV during a CRT procedure.

8. The method of claim 7, further comprising:
identifying a first segment having a highest delay in time to peak velocity;
identifying a minimum level of viability; and
comparing a level of viability of the first segment to the minimum level of viability.

9. The method of claim 7, further comprising:
identifying a minimum level of viability; and
identifying non-viable segments by comparing a level of viability of the segments to the minimum level of viability, the associated recommendation further comprising indicating the non-viable segments as segments to avoid for LV lead placement.

10. The method of claim 7, further comprising:
identifying a first segment having a highest delay in time to peak velocity;
comparing a level of viability of the first segment to a minimum level of viability; and
outputting a recommendation to place the LV lead in the first segment when the first segment exceeds the minimum level of viability and a recommendation to avoid the first segment when the first segment has less than the minimum level of viability.

11. The method of claim 7, further comprising:
dividing the segments into groups comprising at least one of a preferred position for LV lead placement, a sub-optimal position for LV lead placement and a position to avoid for LV lead placement; and
displaying at least one of a bull's eye plot, a list of the segments and a 3D model based on the LV, the segments in each of the groups being indicated differently.

12. The method of claim 7, wherein the delay data comprises a delay in time to peak velocity associated with each of the segments, the method further comprising:
ordering the segments based on the delay data; and
identifying at least one of a degree of viability and a degree of regional function, the associated recommendation being based on the order of segments and the at least one of the degree of viability and the degree of regional function.

13. The method of claim 7, wherein the outputting further comprises displaying a bull's eye plot based on the LV, the associated recommendation with respect to placement of the LV lead comprising indicating the segments on the bull's eye plot in one of different colors, different shades of gray, and different patterns.

14. The method of claim 7, further comprising:
identifying delayed segments; and
comparing a level of viability associated with each of the delayed segments to a minimum level of viability, the associated recommendation further comprising a recommendation to avoid the delayed segments having less than the minimum level of viability.

15. A machine readable medium or media having instructions recorded thereon that are configured to instruct a computer having a processor, a display, and a memory to:
identify left ventricle (LV) segments for placement of an LV lead during a CRT procedure, the LV segments being identified based on a degree of delay and a degree of viability; and
form an output based on the degree of delay and the degree of viability of the LV segments.

16. The machine readable medium or media of claim 15 further having instructions recorded thereon that are configured to instruct the computer to:
identify a minimum level of viability; and
compare the level of viability of each of the LV segments to the minimum level of viability, the output being further based on a relationship between the level of viability and the minimum level of viability for each of the LV segments.

17. The machine readable medium or media of claim 15 further having instructions recorded thereon that are configured to instruct the computer to:
identify a segment within the LV segments having a delay in time to peak velocity; and
compare the degree of viability of the segment to a minimum level of viability, wherein the output comprises a recommendation to place the LV lead in the segment when the segment exceeds the minimum level of viability and a recommendation to avoid the segment when the segment has less than the minimum level of viability.

18. The machine readable medium or media of claim 15 further having instructions recorded thereon that are configured to instruct the computer to:
divide the LV segments into groups based on the degree of delay and the degree of viability associated with each of the LV segments; and
display the groups differently.

19. The machine readable medium or media of claim 15 further having instructions recorded thereon that are configured to instruct the computer to:
identify delayed LV segments based on the degree of delay; and
compare the degree of viability associated with each of the delayed LV segments to a minimum level of viability, the instructions to form an output further comprising indicating that the CRT procedure is not recommended when the degree of viability associated with each of the delayed LV segments is less than the minimum level of viability.

20. The machine readable medium or media of claim 15 further having instructions recorded thereon that are configured to instruct the computer to:
identify a recommendation for each of the LV segments based on the degree of delay and the degree of viability, the recommendations being associated with the placement of the LV lead during the CRT procedure; and
display at least one of a list of the LV segments and the recommendations, a chart associating the LV segments and the recommendations, a bull's eye plot illustrating the LV segments and the recommendations, and a 3D model based on the LV illustrating the LV segments and the recommendations.
